(19) United States
(12) Patent Application Publication
(10) Pub. No.: US 2018/0002452 A1
(43) Pub. Date: Jan. 4, 2018

(54) CHITIN, HYDROLYSATE AND METHOD FOR THE PRODUCTION OF ONE OR MORE DESIRED PRODUCTS BY MEANS OF ENZYMATIC HYDROLYSIS, INCLUDING PRE-TREATMENT WITH AN OXIDISING AGENT

(71) Applicant: YNSECT, Evry Cedex (FR)

(72) Inventors: Nathalie Berezina, Paris (FR); Antoine Hubert, Alfortville (FR); Fabrice Berro, Paris (FR); Jean-Gabriel Levon, Paris (FR); Karine Le Roux, Milly La Foret (FR); Cecilia Socolsky, Paris (FR); Lorena Sanchez, Juvisy (FR); Sophie Laurent, Paris (FR)

(73) Assignee: YNSECT, Evry Cedex (FR)

(21) Appl. No.: 15/541,174

(22) PCT Filed: Dec. 30, 2015
(86) PCT No.: PCT/FR2015/053782

§ 371 Date: Jun. 30, 2017

(30) Foreign Application Priority Data
Dec. 31, 2014 (FR) 1463511

(51) Int. Cl. C08B 37/08; C07K 14/435; C12P 21/06

(52) U.S. Cl. CPC C08B 37/003; C12P 21/06; C07K 14/43563; C07K 14/43577

(57) ABSTRACT

The invention relates to chitin, a hydrolysate and a method for the production of at least one desired product from insects. More specifically, the invention relates to a method for the production of chitin and/or chitosan from insect cuticles, comprising a step in which insect cuticles are treated with an oxidising agent, followed by a step involving the enzymatic hydrolysis of the insect cuticles using a proteolytic enzyme.

The present invention relates to a method for the preparation of at least one product of interest, and more particularly chitin and/or chitosan from insects. More particularly, the invention relates to a method for the preparation of chitin and/or chitosan by enzymatic hydrolysis of insect cuticles. It also relates to a specific chitin and a hydrolysate.

According to the invention, by “chitin” is meant any type of chitin derivative, i.e. any type of polysaccharide derivative comprising N-acetylglucosamine units and D-glucosamine units, in particular the chitin-polypeptide copolymers (sometimes referred to as “chitin/polypeptide composite”).

Chitin is said to be the second most synthesized polymer in the living world after cellulose. In fact, chitin is synthesized by many species in the living world: it constitutes part of the exoskeleton of crustaceans and insects and the lateral wall which surrounds and protects fungi. More particularly, in insects, chitin thus constitutes 3 to 60% of their exoskeleton.

By “chitosan” is meant, according to the present invention, the products of the deacetylation of chitin. The usual limit between chitosan and chitin is determined by the degree of acetylation: a compound having a degree of acetylation less than 50% is called chitosan, and a compound having a degree of acetylation greater than 50% is called chitin.

Chitin and/or chitosan are used in numerous applications: cosmetic (cosmetic composition), medical and pharmaceutical (pharmaceutical composition, treatment of burns, biomaterials, corneal dressings, suture material), dietetics and food processing, technical (filtering, texturizing, flocculating or adsorbent agents in particular for water filtration and purification), etc. In fact, chitin and/or chitosan are materials that are biocompatible, biodegradable and non-toxic.

Traditionally, chitin is extracted chemically from crustaceans, from cephalopods, but also, more exceptionally, from fungi. The chemical route uses large quantities of reagents (such as hydrochloric acid, sodium hydroxide and bleaching agents), which have the effect of denaturing the structure of chitin such as it exists in the natural state, for example as present in the shell of crustaceans. Moreover, most of the chemical reagents are harmful to humans and the environment and generate large volumes of effluents that have to be treated. Finally, chitin and/or chitosan originating from crustaceans can generate allergic reactions in sensitive persons.

Another route for extraction of chitin is the enzymatic route. This route is considered to be milder, thus making it possible to better preserve the chitin and/or chitosan. However, the chitin obtained by this route is of a brownish colour, requiring purification steps in order to obtain a marketable powder, i.e. of white colour. The existing methods therefore generally comprise one or more steps for removing the impurities from chitin, such as a step of demineralization with acid, carried out prior to enzymatic hydrolysis, and/or a step of bleaching the chitin with an oxidizing agent, carried out after enzymatic hydrolysis. These two steps for the purification of chitin unfortunately have the effect of altering the chemical structure of chitin.

Work undertaken by the inventors demonstrated that it is possible to obtain chitin that is both purer and has a structure closer to the original structure of chitin by treating insect cuticles with an oxidant before carrying out enzymatic hydrolysis.

The invention therefore relates to a method for the production of chitin and/or chitosan starting from insect cuticles, comprising the following steps:

(i) treating insect cuticles with an oxidizing agent, then

(ii) enzymatic hydrolysis of the insect cuticles with a proteolytic enzyme.

By “insects” is meant insects at any stage of development, such as an adult, larval or nymph stage. Preferably, the insects used in the method according to the invention are edible.

More particularly, the insects can be selected from the group constituted by the Coleoptera, Diptera, Lepidoptera, Orthoptera, Isoptera, Hymenoptera, Blattoptera, Hemiptera, Heteroptera, Ephemeroptera and Mecoptera, preferably from the Coleoptera, Diptera, Orthoptera and Lepidoptera.

Preferably, the insects are selected from the group constituted by Tenebrio molitor, Hermetia illucens, Galleria mellonella, Alphitobius diaperinus, Zophobas morio, Blattera fusca, Tribolium castaneum, Rhynchophorus ferrugineus, Musca domestica, Chrysomya megacephala, Locusta migratoria, Schistocerca gregaria, Acheta domesticus and Samia ricini.

More preferably, the insects are selected from the group constituted by Tenebrio molitor, Hermetia illucens, Galleria mellonella, Alphitobius diaperinus, Zophobas morio, Blattera fusca, Musca domestica, Chrysomya megacephala, Locusta migratoria, Schistocerca gregaria, Acheta domesticus and Samia ricini, and even more preferably, T. molitor.

One or more insect species can be used in the method according to the invention, preferably a single insect species. If several species are used, advantageously two closely related species will be selected, for example Hermetia illucens and Musca domestica.

The insects are preferably reared, rather than taken from nature. For example, the insects are reared in an insect farm. Breeding the insects in a special farm makes it possible not only to control and eliminate the risks associated with insect-borne diseases, but also to limit the risks associated with the toxicity of food products derived from insects due for example to the presence of insecticides. Moreover, farming makes it possible to control the quality of the supply of insects and limit the costs of supply.

By “treatment of insect cuticles” is meant not only treatment of the cuticles once they have been separated from the insects, but also treatment of the cuticles, including some or all of the other constituents of the insect, including treatment of the insect in its entirety. In fact, it is possible to apply the method according to the invention to the whole insect, such as insects that have been ground, or else to only a part of the insects comprising the cuticles, for example after extraction, such as insects that have been ground and then pressed in order to extract a press cake rich in cuticles, or such as exuviae/molted cuticles separated naturally, and collected by a suitable method.

The cuticle is the outer layer (or exoskeleton) secreted by the epidermis of the insects. Generally it is formed from three layers:
the epicuticle, which is the thinnest, outermost layer of the cuticle (less than 4 μm); this layer is impermeable to water and comprises a layer of water-repelling wax, as well as a smaller amount of proteins and chitin;
the exocuticle, which is the intermediate layer of the cuticle; it consists essentially of proteins that have been hardened by tanning, and are responsible for the rigidity of the cuticle, chitin, and optionally melanin; and
the endocuticle, which is a thin, flexible layer, constituted by a mixture of proteins and chitin.

Preferably, in the method according to the invention, the oxidizing agent used in the treatment of the cuticles is selected from the group constituted by hydrogen peroxide, potassium permanganate, ozone and sodium hypochlorite, even more preferably hydrogen peroxide.

Advantageously, when the oxidizing agent is hydrogen peroxide, the quantity of this agent introduced for treating the insect cuticles is such that the hydrogen peroxide content is between 1 and 33% by weight based on the total weight of insects, preferably between 2 and 12% by weight based on the total weight of insects, preferably of the order of 6% by weight.

In the present application, the ranges of values are understood to be inclusive. Moreover, when “approximately” or “of the order of” precedes a number, this is equivalent to plus or minus 10% of the value of this number.

Preferably, treatment of the insect cuticles with the oxidizing agent is carried out in the presence of water, such as fresh water. Advantageously, the quantity of water used in the treatment of the cuticles is determined as follows: the ratio of the volume of water in mL to the weight of insect in g is preferably comprised between 0.3 and 10, more preferably between 0.5 and 5, even more preferably between 0.7 and 3, even more preferably of the order of 1. It should be noted that this ratio also corresponds to the ratio of the weight of water to the weight of insect, the density of water being 1.0 g/mL under normal temperature and pressure conditions.

Treatment of the insect cuticles is followed by enzymatic hydrolysis.

According to the invention, the enzymatic hydrolysis is carried out with at least one proteolytic enzyme, preferably a protease. In the present application, the names or suffixes “peptidase” and “protease” are used indiscriminately to denote an enzyme causing lysis of a peptide bond of the proteins. Advantageously, this is carried out for a time of from 4 to 8 h, preferably for 4 to 5 h, at a temperature from 40 to 60° C., preferably 45 to 55° C. and at a pH comprised between 6 and 8, preferably between 6.5 and 7.5.

Enzymatic hydrolysis can be carried out with a single protease or alternatively with a mixture of enzymes containing at least one protease, more preferably a mixture of enzymes containing several proteases, such as a mixture containing an endoprotease and an exoprotease, or a protease and a polysaccharase.

Preferably, the protease is selected from the group constituted by the aminopeptidases, metallocarboxypeptidases, serine endopeptidases, cysteine endopeptidases, aspartic endopeptidases, metalloendopeptidases.

Advantageously, the enzymes can be selected from the following:

| | | EC | | | |
|---|---|---|---|---|---|
| Enzyme(s) | Class | number | Supplier | Town | Country |
| Flavourzyme | Amino- | EC | Novozyme | Bagsvaerd | Denmark |
| | peptidases | 3.4.11.1 | | | |
| Fungal | | EC | BioCat | Troy | United |
| protease 500 | | 3.4.11.1 | | | States |
| Kojizyme | | EC | Novozyme | Bagsvaerd | Denmark |
| | | 3.4.11.1 | | | |
| Protex P | Serine | EC 3.4.21 | Genencor | Leiden | The |
| | endopeptidases | | International | | Netherlands |
| | | | B.V. | | |
| Chymotrypsin | | EC | Novozyme | Bagsvaerd | Denmark |
| | | 3.4.21.1 | | | |
| Protamex | | EC 3.4.21 | Novozyme | Bagsvaerd | Denmark |
| Elastase | | EC | Novozyme | Bagsvaerd | Denmark |
| | | 3.4.21.14 | | | |
| Trypsin | | EC | Novozyme | Bagsvaerd | Denmark |
| | | 3.4.21.36 | | | |
| Alcalase | | EC | Novozyme | Bagsvaerd | Denmark |
| | | 3.4.21.4 | | | |
| Papain | Cysteine | EC | BioCat | Troy | United |
| | endopeptidases | 3.4.22.2 | | | States |
| Bromelain | | EC | BioCat | Troy | United |
| (ananase) | | 3.4.22.32 | | | States |
| Prolyve NP | Aspartic | EC 3.4.23 | Lyven | Colombelles | France |
| Pepsin | endopeptidases | EC | Sigma | Saint- | France |
| | | 3.4.23.1 | Aldrich | Quentin- | |
| | | | | Fallavier | |
| Neutral | Metallo- | EC | BioCat | Troy | United |
| protease | endopeptidase | 3.4.24.28 | | | States |
| Protex 50FP | Endopeptidase | EC 3.4.21 | Genencor | Leiden | The |
| | | | International | | Netherlands |
| | | | B.V. | | |
| Pancrealyve | Exo & endo | n.a.* | Lyven | Colombelles | France |
| | peptidase | | | | |
| | (cocktail of | | | | |
| | proteases + | | | | |
| | amylases) | | | | |
| Izyme BA | Aspartic | EC 3.4.23 | Novozyme | Bagsvaerd | Denmark |
| | protease | | | | |
| Sumizyme | Enzyme cocktail | n.a.* | Takabio - | Aichi | Japan |
| | | | Shin Nihon | | |
| Neutrase | Endoprotease | EC 3.4.24 | Novozyme | Bagsvaerd | Denmark |
| | Zn base of β | | | | |
| | amyloliquefaciens | | | | |
| Novozyme | Protease | n.a.* | Novozyme | Bagsvaerd | Denmark |
| 37071 | | | | | |

*n.a.: not applicable

Advantageously, the enzyme used in the hydrolysis is an aspartic endopeptidase, such as Prolyve NP. This type of enzyme makes it possible to obtain very good results in terms of purity of the chitin obtained, especially when this type of enzyme is applied in the hydrolysis of a press cake obtained from Coleoptera and more particularly from T. molitor.

The enzyme or the mixture of enzymes is introduced in a quantity ranging from 0.2 to 10% by weight of estimated dry matter, preferably from 0.4 to 8% by weight and more preferably from 0.5 to 2%. By “weight of estimated dry matter” is meant more particularly the weight of dry matter from insects or insect part(s), such as can be estimated when entering the enzymatic hydrolysis step.

In terms of enzymatic activity, the quantity of enzyme or enzyme mixture introduced is equivalent to an activity comprised between 2000 and 5000 SAPU (“Spectrophotometric Acid Protease Unit”, described in Example 4 below), preferably between 3000 and 4000 SAPU, per 100 g wet weight, with a water content from 30 to 70%, of substrate to be transformed, i.e. of insect or hydrated insect part(s).

Advantageously, the enzymatic hydrolysis step is carried out in the presence of water, such as fresh water. The quantity of water used in the enzymatic hydrolysis is determined as follows: the ratio of the volume of water in mL to the weight of insect in g is preferably comprised between 0.3 and 10, more preferably between 0.5 and 5, even more preferably between 0.7 and 3, even more preferably of the order of 1.

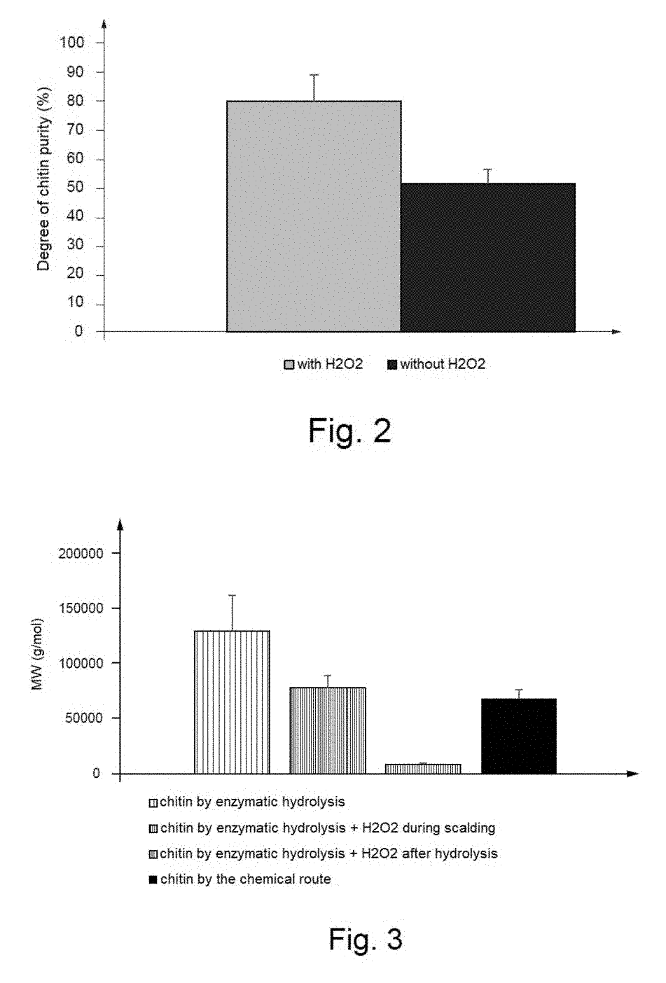

The method according to the invention makes it possible to obtain a chitin having a degree of purity (or gravimetric purity) comprised between 55 and 90%, preferably between 60 and 85%, even more preferably of the order of 80% (see Example 2 and FIG. 2).

In addition, the size of the chitin obtained by the method according to the invention, namely of the order of 80,000 g/mol in Example 3 below, is very close to that which exists in the natural state in the insect cuticle.

Advantageously, the method according to the invention comprises a step of killing the insects. This killing step is carried out prior to enzymatic hydrolysis. The killing step can be carried out by conventional methods in the farming of cold-blooded animals and/or animals of small size (crustaceans, fish, snails, etc.), such as cold (freezing), heat (scalding), oxygen deprivation, etc.

Preferably, killing is carried out by scalding. Scalding kills the insects while lowering the microbial load (reducing the risk of deterioration and health risk) and inactivating the internal enzymes of the insects which can trigger autolysis, and thus a rapid browning thereof. Moreover, scalding is carried out in such a way as to cause death as quickly as possible, in order to respect animal welfare, and according to scientific recommendations.

Preferably, the scalding step is carried out in water, such as fresh water, at a temperature from 95 to 105° C., preferably of the order of 100° C. and for a time from 2 to 20 min, preferably 5 to 15 min.

The quantity of water introduced in this scalding step is determined as follows: the ratio of the volume of water in mL to the weight of insect in g is preferably comprised between 0.3 and 10, more preferably between 0.5 and 5, even more preferably between 0.7 and 3, even more preferably of the order of 1.

Preferably, treatment of the insect cuticles with the oxidizing agent can be carried out concomitantly with scalding and/or after the scalding step, more preferably concomitantly with scalding.

Alternatively, killing can be carried out by blanching.

Blanching has the same advantages as scalding as mentioned above.

Preferably, the blanching step is carried out with steam and/or with water at a temperature between 80° C. and 130° C., preferably between 90° C. and 120° C.

Treatment of the insect cuticles with the oxidizing agent can be carried out concomitantly with blanching and/or after the blanching step, preferably concomitantly with blanching.

When treatment of the insect cuticles is carried out during scalding or blanching, the oxidizing agent is added to the water used for scalding or blanching the insects.

Advantageously, the method according to the invention also comprises a step of grinding the insects.

Preferably, this grinding step takes place after the scalding or blanching step. This grinding step has the aim of reducing the insects to particles in order to facilitate access of the enzymes to the substrate during enzymatic hydrolysis. This step also makes it possible, when it is followed by a pressing step, to facilitate removal of the press juice and isolation of the solid matter.

Grinding can advantageously be carried out with a mixer-grinder, such as a knife mill.

Preferably, at the end of grinding, the size of the particles of insects is less than 1 cm (largest particle size observable using a microscope), preferably less than 0.5 cm, even more preferably a size comprised between 300 μm and 0.5 cm, preferably 500 μm and 0.5 cm and even more preferably between 500 μm and 1 mm.

In order to facilitate the grinding, a quantity of water can be added. This quantity of water is determined as follows: the ratio of the volume of water in mL to the weight of insect in g is preferably comprised between 0.3 and 10, more preferably between 0.5 and 5, even more preferably between 0.7 and 3, even more preferably of the order of 1.

Optionally, treatment of the insect cuticles with the oxidizing agent can be carried out concomitantly with grinding. In this case, the oxidizing agent is added to the water used for grinding. Alternatively, treatment of the cuticles can be carried out during scalding, grinding and/or in a specific treatment step of the insect cuticles.

The method according to the invention can further comprise, preferably before enzymatic hydrolysis, a pressing step. Although this pressing step can be carried out before the grinding step, it is advantageously carried out just after the grinding step. This pressing step has the objective of removing fat-rich press juice and enriching the press cake with substrate for hydrolysis.

Advantageously, enzymatic hydrolysis can be followed by a step of heat inactivation for the purpose of inactivating the enzyme or the enzyme mixture used in enzymatic hydrolysis.

At the end of a method according to the invention, the chitin can be recovered by pressing or centrifugation of the enzymatic hydrolysis reaction mixture. At this stage, a chitin co-product of interest is also recovered, namely a hydrolysate.

By “hydrolysate” is meant a product that comprises proteins, hydrolysed proteins, peptides, amino acids and/or other compounds derived from proteins, obtainable by enzymatic hydrolysis of proteins.

The invention also relates to a hydrolysate, such as a hydrolysate obtainable as a co-product of enzymatic hydrolysis by any one of the methods according to the invention.

The hydrolysate according to the invention has at least any one of the following characteristics:
Ash content ≦10% by weight based on the total weight of dry matter, preferably ≦8%
Ash content ≦5% when the hydrolysate is prepared from vegetarian insects
Content of water-soluble proteins of size >12,400 g/mol ≦50%, preferably ≦43%
Protein content ≧40%
Protein content ≧46% when the hydrolysate is prepared from non-flying insects
Lipid content ≦52%
Pepsin digestibility ≧94%
Relative abundance of at least any 5 amino acids selected from Asp, Glu, Ala, Gly, Leu, Pro, Tyr, Val, Lys >6%
Relative abundance of at least any 2 amino acids selected from Asp, Glu, Ala, Leu, Pro, Tyr, Val >8%

By “vegetarian insect” is meant an insect that does not have animal proteins in its usual diet. By way of an example of vegetarian insects, the Coleoptera, Lepidoptera or Orthoptera may be mentioned.

By “flying insect” is meant an insect that is capable of flying when adult, in contrast to an insect called “non-flying”. By way of an example of flying insects, the Lepidoptera or Diptera may be mentioned. By way of an example of non-flying insects, certain Coleoptera or the Orthoptera may be mentioned.

More particularly, by “water-soluble proteins” is meant, among the proteins (or crude proteins), those that are soluble in an aqueous solution the pH of which is comprised between 6 and 8, advantageously between 7.2 and 7.6.

Preferably, the aqueous solution is a buffer solution the pH of which is comprised between 6 and 8, advantageously between 7.2 and 7.6.

Preferably, the buffer solution is a phosphate buffered NaCl solution, the pH of which is equal to 7.4±0.2.

More particularly, the size of the water-soluble proteins is measured by the following method:

100 mg of sample was introduced into 10 mL of phosphate/NaCl buffer (pH 7.4, 0.137 mM). The sample was stirred for one minute (vortex), centrifuged at 900 g for 1 min and then filtered through a 0.45 μm membrane. The analysis was carried out on a steric exclusion chromatography system, with the Nucleogel GFC-300 column, the eluent used is phosphate/NaCl buffer (pH 7.4, 0.137 mM), the flow rate is 1.0 mL/min, with detection by a UV detector at 280 nm.

The hydrolysate according to the invention comprises at least 40% proteins, at most 10% and preferably at most 8% ash, and a content of water-soluble proteins having a size greater than 12,400 g/mol less than 50%, preferably less than 43%.

All the units and methods of measurement of the characteristics stated above are described in the examples, and more particularly in Example 4.

Preferably, the hydrolysate has all the properties stated above.

It will be noted in particular that the hydrolysate according to the invention can be distinguished from any other hydrolysate by its content of glucosamine and/or a derivative thereof (preferably N-acetyl-glucosamine), more particularly a content greater than or equal to 0.01%, preferably greater than or equal to 0.08% by weight based on the total weight of dry matter of the hydrolysate.

This hydrolysate can advantageously be supplemented with additives for balancing its nutritional profile to make it suitable for different types of animals.

The hydrolysate can be concentrated and then dried to obtain a dried hydrolysate. Alternatively, the hydrolysate can be in liquid form. These hydrolysates can be used as a foodstuff or a food ingredient in particular for animals, or alternatively they can be treated, for example to isolate amino acids.

A preferred embodiment of a method according to the invention is described in more detail below.

In particular, this preferred embodiment describes various advantageous steps for a method according to the invention, such as steps of mild purification of the chitin: a second pressing, washing operations, optional filtration and drying.

Finally, as chitin is generally marketed in the form of powder, a second grinding can also be carried out. The latter can also be carried out to promote the deacetylation reaction, for preparing chitosan from chitin. The conditions of the deacetylation reaction are described more fully in step 10 of the preferred embodiment described in detail below.

A particularly advantageous method for the production of chitin from insect cuticles comprises the following steps:
a) killing the insects,
b) grinding the insects, grinding optionally being preceded or followed by a pressing step,
c) enzymatic hydrolysis of the insect cuticles with a proteolytic enzyme,
d) recovery of the chitin, the insect cuticles being treated with an oxidizing agent before step c).

The preferred embodiments of the various steps a) to d) as well as treatment with the oxidizing agent are as stated above or in the corresponding step in the preferred embodiment below.

The invention also relates to a chitin, such as a chitin obtainable by a method according to the invention. This chitin has a structure similar to the chitin present in the natural state in the insect cuticle.

The chitin according to the invention has at least any one of the following characteristics:
Ash content ≦3.5%, preferably ≦3% by weight based on the total weight of dry matter
Ash content ≦2.5% when the chitin is prepared from vegetarian insects
Ash content ≦1.7% when the chitin is prepared from T. molitor
Lipid content ≦29%
Lipid content ≦19% when the chitin is prepared from vegetarian insects
Lipid content ≦8.5% when the chitin is prepared from T. molitor
Total amino acids ≦55%, preferably ≦53%
Total amino acids ≦30% when the chitin is prepared from flying insects
Relative abundance of at least any 3 amino acids selected from Ala, Gly, Leu, Pro, Ser, Tyr, Val ≦10%
Relative abundance of Leu, Pro, Val ≦10% when the chitin is prepared from T. molitor
Colorimetric purity ≧44%
Colorimetric purity ≧48% when the chitin is prepared with Prolyve
Purity by difference ≧35%
Purity by difference ≧38% when the chitin is prepared from T. molitor
Purity by difference ≧50% when the chitin is prepared from flying insects

The chitin according to the invention comprises an amino acid content less than or equal to 55%, preferably less than or equal to 53%, an ash content less than or equal to 3.5%, preferably less than or equal to 3%, and a purity by difference greater than or equal to 35% or a colorimetric purity greater than or equal to 44%.

Preferably, the chitin has all the properties stated above.

All the units and methods of measurement of the characteristics stated above are described in the examples, and more particularly in Example 4.

A particularly advantageous method for the production of chitosan from insect cuticles comprises the following steps:
a) killing the insects,
b) grinding the insects, grinding optionally being preceded or followed by a pressing step,
c) enzymatic hydrolysis of the insect cuticles with a proteolytic enzyme,
d) recovery of the chitin,
e) deacetylation of the chitin recovered,
f) recovery of the chitosan, the insect cuticles being treated with an oxidizing agent before step c).

The preferred embodiments of the various steps a) to f), as well as of the treatment with the oxidizing agent are as stated above or in the corresponding step in the preferred embodiment below.

The invention finally relates to a chitosan obtainable by a method according to the invention.

The chitin and/or chitosan obtainable by a method according to the invention can advantageously be used in various applications:
in cosmetic, pharmaceutical, nutraceutical or dietetic compositions,
as biomaterials for treating burns, as second skin, for making corneal dressings or suture materials,
as filtering, texturizing, flocculating and/or adsorbent agents in particular for water filtration and purification.

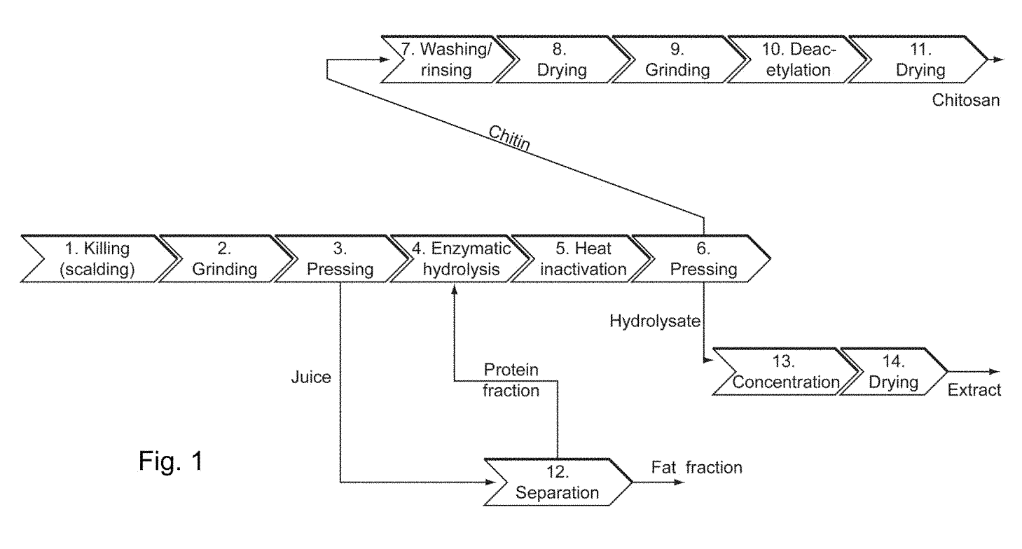

According to a preferred embodiment of the invention, the method comprises the following steps, described schematically in FIG. 1. It should be noted that certain steps are indicated as optional in this preferred embodiment.

Step 1: Killing the Insects

This killing step 1 makes it possible to kill the insects while reducing the microbial load (reducing the risk of deterioration and health risk) and by inactivating the internal enzymes of the insects which can trigger autolysis, and thus a rapid browning thereof.

Killing can be carried out by scalding.

The insects, preferably larvae, are thus scalded with water for 2 to 20 min, preferably 5 to 15 min. Preferably, the water is at a temperature comprised between 95 and 105° C., preferably 100° C.

The quantity of water introduced in this scalding step 1 is determined as follows: the ratio of the volume of water in mL to the weight of insect in g is preferably comprised between 0.3 and 10, more preferably between 0.5 and 5, even more preferably between 0.7 and 3, even more preferably of the order of 1.

Alternatively, killing can be carried out by blanching. Preferably, the insects are blanched with steam (steam nozzles or bed) at a temperature comprised between 80 and 130° C., preferably between 90 and 120° C., more preferably between 95 and 105° C., even more preferably 98° C. or with water at a temperature comprised between 95 and 105° C., preferably 100° C. (by spray nozzles) or in mixed mode (water+steam) at a temperature comprised between 80 and 130° C., preferably between 90 and 120° C., more preferably between 95 and 105° C. The residence time in the blanching chamber is comprised between 1 and 15 minutes, preferably between 3 and 7 min.

In this step, it is also possible to treat the insect cuticles using scalding or blanching water comprising the oxidizing agent according to the details indicated in the intermediate step below.

Intermediate Step (Optional): Treatment of Cuticles with the Oxidizing Agent

A special step of treatment of the cuticles with the oxidizing agent can be incorporated in the method. Advantageously, this intermediate step of treatment of the cuticles is carried out between the killing step 1 and the grinding step 2.

This intermediate step is preferably carried out with an oxidizing agent selected from the group constituted by hydrogen peroxide (H₂O₂), potassium permanganate (KMnO₄), ozone (O₃) and sodium hypochlorite (NaClO), more preferably hydrogen peroxide.

According to a first embodiment, at the end of step 1, when the latter is carried out by scalding, the oxidizing agent is introduced directly into the scalding tank, after optional cooling of the scalding water to a temperature of the order of 40 to 60° C., preferably of the order of 50° C.

The hydrogen peroxide as marketed is usually in the form of an aqueous solution, for example a solution at 30% by weight based on the total weight of water.

The quantity of hydrogen peroxide introduced for the treatment is such that the hydrogen peroxide content is comprised between 1 and 33% by weight based on the total weight of insects, preferably 2 to 12% by weight based on the total weight of insects, preferably of the order of 6% by weight.

According to a second embodiment, the insects are removed from the scalding tank, sieved and reintroduced into a tank.

The hydrogen peroxide is then introduced into the tank in the form of a dilute aqueous solution, the hydrogen peroxide content then being comprised between 1 and 33% by weight based on the weight of water, preferably 2 to 12% by weight based on the weight of water, and preferably of the order of 6% by weight.

Step 2: Grinding

The insects are removed from the scalding or treatment tank or from the blanching chamber, then they are sieved, and placed in a grinder, such as a knife mill, making it possible to reduce the insects to particles.

In order to facilitate the grinding, a quantity of water can be added. This quantity of water is similar to that introduced during the scalding step 1: the ratio of the volume of water in mL to the weight of insect in g is preferably comprised between 0.3 and 10, more preferably between 0.5 and 5, even more preferably between 0.7 and 3, even more preferably of the order of 1. It is also possible to keep the scalding water and/or the water resulting from the intermediate step in order to carry out this step. This water is likely to contain the oxidant. In this case, treatment of the cuticles can take place during the scalding step 1 and the grinding step 2 or during the intermediate step of treatment of the cuticles and during the grinding step.

Preferably, on completion of the grinding, the size of the insect particles is less than 1 cm (largest particle size observable using a microscope), preferably less than 0.5 cm.

Preferably, on completion of the grinding, the size of the insect particles is comprised between 300 μm and 0.5 cm, more preferably between 500 μm and 0.5 cm and even more preferably between 500 μm and 1 mm.

It is not necessary to excessively reduce the size of the particle, for example to a size less than 250 μm.

This grinding step 2 promotes access of the enzymes to their substrate.

In this step, it is possible to introduce the oxidizing agent into the grinding mill in order to treat the cuticles according to the methods indicated in the preceding intermediate step.

When treatment of the cuticles is not carried out concomitantly with grinding, during this step it is possible to add antioxidant additives that are commonly used for product preservation and stability.

Step 3 (Optional): Pressing

The wet paste originating from the grinding step 2 is then placed in a press according to a procedure which makes it possible to press and separate a juice comprising both a fat fraction and a protein fraction.

If the wet paste from the grinding step 2 was obtained with water comprising the oxidant, it can be advantageous to eliminate at least a part of this oxidant before the pressing step 3.

This pressing step 3 can optionally be carried out before the grinding step 2 starting from scalded insects.

This pressing step 3 therefore makes it possible to obtain a press juice and a press cake.

Step 4: Enzymatic Hydrolysis

The wet paste originating from the grinding step 2 or the press cake originating from the pressing step 3 is placed in a hydrolysis tank with water.

Optionally, and as will be described below, the protein fraction originating from the separating step 12 can be reintroduced in this enzymatic hydrolysis step 4, by mixing it with the press cake.

Optionally, and in the case when the scalding water does not contain oxidant, the scalding water can be recovered and reintroduced in the hydrolysis step. In fact, this water contains insect fractions that have been solubilized by the action of this scalding, and by using the latter in the hydrolysis it is possible to reduce the losses. Optionally, this water from scalding can be defatted, as certain waxes can have dissolved in the water.

The quantity of water introduced in this enzymatic hydrolysis step 4 is similar to that introduced in the scalding step 1: the ratio of the volume of water in mL to the weight of insect in g is preferably comprised between 0.3 and 10, more preferably between 0.5 and 5, even more preferably between 0.7 and 3, even more preferably of the order of 1.

Enzymatic hydrolysis is carried out with a protease, such as a commercial protease, for 4 to 8 h, more particularly for 4 to 5 h, at a pH from 6 to 8, more particularly from 6.5 to 7.5, at a temperature from 40 to 60° C., more particularly from 45 to 55° C.

The quantity of enzymes introduced in the hydrolysis step is less than 10% by weight based on the estimated total weight of dry matter entering hydrolysis, preferably less than 6%, more preferably of the order of 2%.

Proteolytic hydrolysis results in the production of a soluble phase containing the peptides, glucosamines and oligochitins and a solid residue formed from chitin, mainly chitin-polypeptide copolymer.

Step 5: Heat Inactivation

In order to stop the activity of the enzymes of the reaction and stabilize the soluble phase of the hydrolysis, heat inactivation is carried out by heating this juice between 80 and 105° C. for 10 to 25 min, preferably 15 to 20 minutes. According to one procedure, this heat inactivation step 5 is carried out according to the usual sterilization techniques of the agri-food industry. According to another procedure, enzyme inactivation is carried out by heating under IR or UV radiation, or by microwave heating.

Step 6: Pressing

The solid residue, predominantly composed of chitin, is recovered and then pressed in a press for maximum draining of this residue, in order to reinject this pressate into the soluble phase. The pressed residue thus formed consists essentially of chitin, mainly in the form of chitin-polypeptide copolymer.

Steps 7 and 8 (Optional): Washing and Drying

The solid residue is then washed, filtered, washed again and then dried by the conventional technologies known to a person skilled in the art.

Advantageously, the drying system is designed to protect the structure of the chitin-polypeptide copolymer: the hydrometry, ventilation and composition of the air are controlled. Advantageously, drying can be carried out in a ventilated stove at a temperature from 60 to 80° C., preferably of the order of 70° C.

Optionally, these steps can comprise a final delipidation step: the solid residue is treated with HCl in order to remove the last lipid residues, in particular the cuticular waxes.

The next steps 9 to 11 are for transforming the chitin to chitosan and therefore are only used when the desired product is chitosan.

Step 9 (Optional): Grinding

The dried solid residue, comprising predominantly chitin, is then ground, for example in a cutting (knife) mill.

The production of chitosan from chitin, by the deacetylation reaction, largely depends on the size of the chitin particles. Thus, very fine grinding of the dried solid residue before deacetylation makes it possible to increase the yields and the rate of the deacetylation reaction significantly, as shown in Table 1 below:

TABLE 1
Efficiency of deacetylation according to previous
grinding of chitin

| | Grinding | Grinding | Grinding | Grinding |
|---|---|---|---|---|
| | 30 s | 45 s | 60 s | 120 s |
| 50% of the | <174 μm | <117 μm | <95 μm | <67 μm |
| particles | | | | |
| 90% of the | <310 μm | <244 μm | <157 μm | <159 μm |
| particles | | | | |
| DA* | 99% | 90% | 85% | 80% |

*Measurement of the Degree of Acetylation DA

The conditions of the deacetylation carried out in the test reported in Table 1 are as follows: reaction time 4 h, 100° C., NaOH in aqueous solution at 30 vol %, in a ratio of estimated chitin:NaOH solution equal to 1:20.

Consequently, the solid residue is preferably ground to a particle size less than 200 μm, more preferably less than 160 μm.

Step 10: Deacetylation

The solid residue, optionally ground in step 9, is then placed in a reactor, to which concentrated sodium hydroxide solution is added, for 4 to 24 h, and preferably 6 to 18 h. Sodium hydroxide in aqueous solution at a content ranging from 30% to 40% is added at a ratio of weight in g of ground chitin/volume in mL of sodium hydroxide in aqueous solution comprised between 1:50 and 1:10, preferably of the order of 1:20. The tank is then heated, the deacetylation temperature being between 80 and 150° C., preferably between 90 and 120° C. and more preferably at 100° C.

Chitosan is thus obtained in powder form.

The chitosan can then undergo any operation known to a person skilled in the art allowing it to be functionalized, in particular by adding radicals (carboxylation, hydroxylation, etc.).

Step 11 (Optional): Drying

The chitosan powder is then dried at between 30 and 80° C., preferably between 50 and 70° C. and preferably at approximately 60° C., in order to obtain a powder having a dry matter content greater than 85%, more particularly greater than 90%.

The next steps are for recovering a fat fraction and a protein fraction from the juice obtained in the (optional) pressing step 3 and therefore are only used when the pressing step 3 has been used and when such recovery is desired.

Step 12: Separation

The press juice undergoes one or more separating steps, in order to separate the fat fraction (insect oils) from the protein fraction (insect haemolymph proteins). These steps can be carried out by any oil separation technology well known to a person skilled in the art, such as centrifugation, decanting, separation by reverse osmosis, ultrafiltration, supercritical CO₂, etc., or a combination of several of these technologies.

Separation of the fat fraction can be complex, in view of the presence of oils of very varied compositions in insects, and the fatty acids can have short chains (C2-C5) as well as very long chains (for example, for waxes: >C25). Raising the temperature above the melting point of these oils (approximately 38° C.) during centrifugation makes it possible to solubilize this cream and facilitate separation of the fat fraction from the rest of the juice. The centrifugate then undergoes decanting according to a procedure (of the decanter or Tricanter type), for best possible separation of the oils and proteins.

These steps thus make it possible to obtain a fat fraction.

Once separated from the fat fraction, the protein fraction can be mixed with the press cake originating from the pressing step 3 just before the hydrolysis step 4. In fact, often more than 20% of the proteins are lost in the juice in the pressing step 3, hence the benefit of recovering this fraction and subjecting it to the hydrolysis step.

Step 13 (Optional): Concentration

According to one procedure, concentration is carried out by vacuum evaporation of the aqueous part. The concentrate has a dry extract greater than 10%, preferably greater than 20%. This operation facilitates drying, and additives commonly used for product preservation and stability can be added in this step.

Step 14 (Optional): Drying

The concentrate is finally dried by technologies that are known to a person skilled in the art, for example spraying/atomization (“spray-drying”), which makes it possible to obtain an extract, i.e. a dry powder of concentrate rich in peptides and glucosamines, the glucosamines in particular originating from the partial hydrolysis of chitin by H₂O₂ (essentially).

Other features and advantages of the invention will become clear from the following examples, given by way of illustration, with reference to the figures, which represent respectively:

FIG. 1 is a flow chart of a preferred embodiment of a method according to the invention,

FIG. 2 is a diagram comparing the degree of purity for the chitin obtained by an enzymatic method with and without hydrogen peroxide,

FIG. 3 is a diagram illustrating the influence of the method of extraction (enzymatic or chemical) and treatment with an oxidizing agent on the chitin obtained.

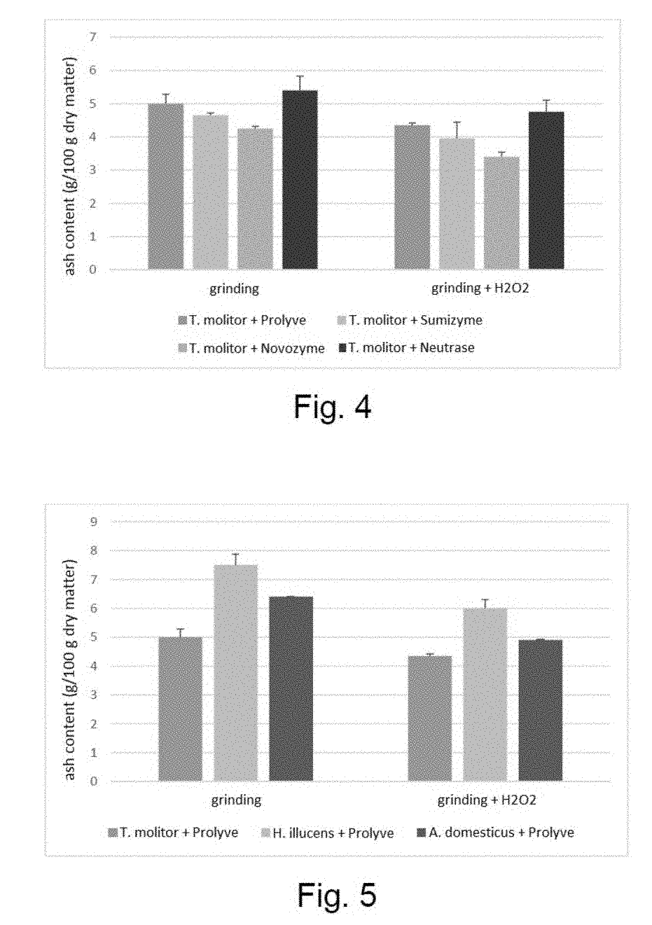

FIG. 4: Effect of the oxidizing agent on the ash content in the hydrolysate-different enzymes,

FIG. 5: Effect of the oxidizing agent on the ash content in the hydrolysate-different insects,

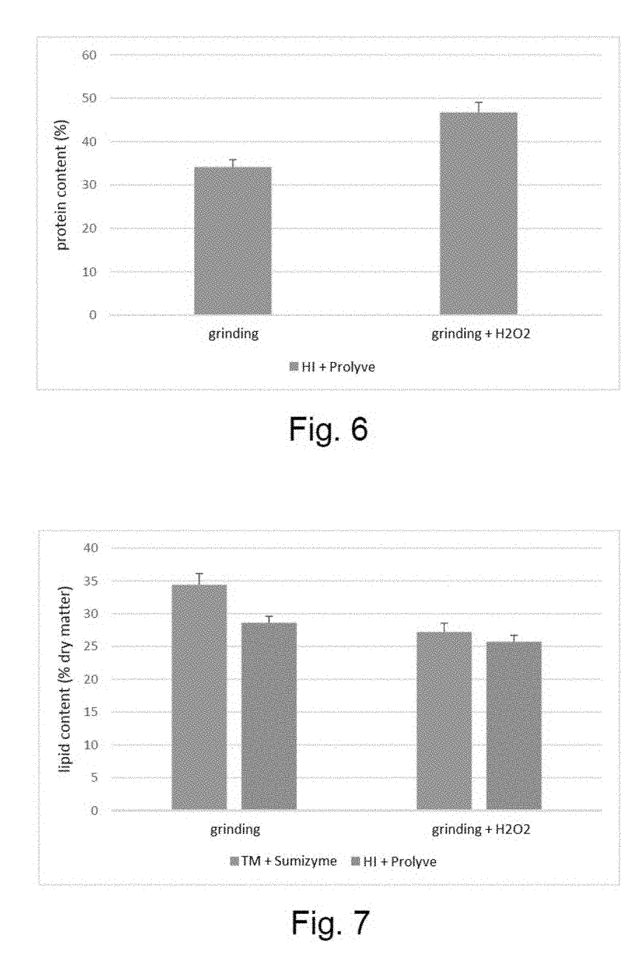

FIG. 6: Effect of the oxidizing agent on the protein content in H. illucens,

FIG. 7: Effect of the oxidizing agent on the lipid content in the hydrolysate,

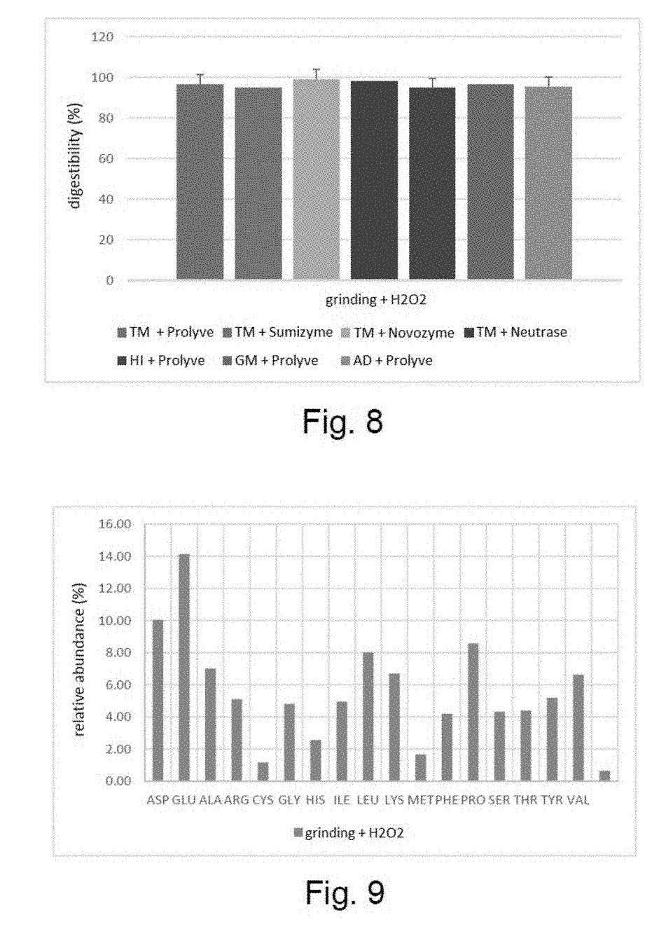

FIG. 8: Effect of the oxidizing agent on the digestibility of the hydrolysate,

FIG. 9: Amino acid composition of the hydrolysate from a method with an oxidizing agent: TM+Prolyve,

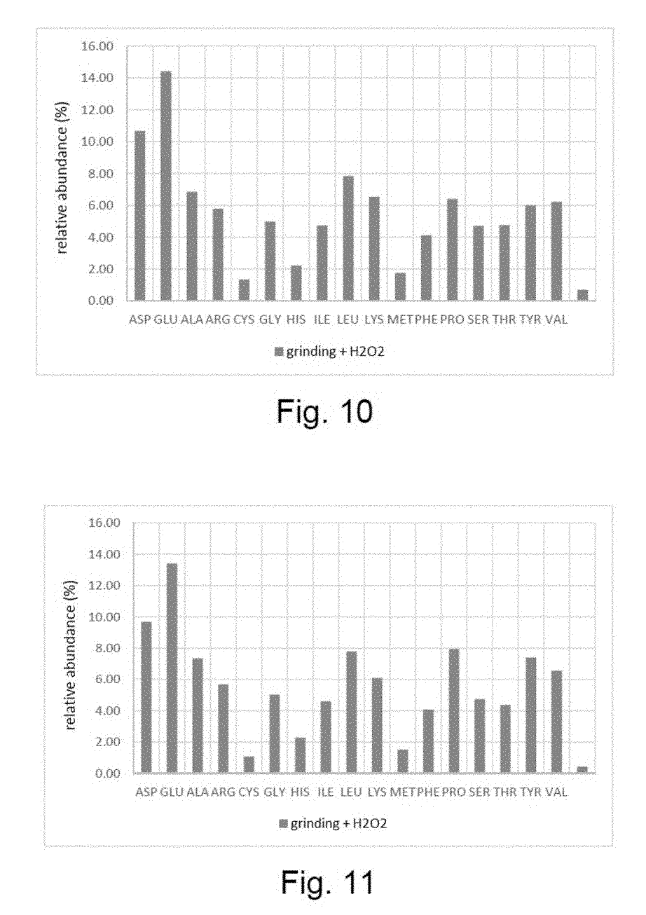

FIG. 10: Amino acid composition of the hydrolysate from a method with an oxidizing agent: TM+Sumizyme,

FIG. 11: Amino acid composition of the hydrolysate from a method with an oxidizing agent: TM+Novozyme,

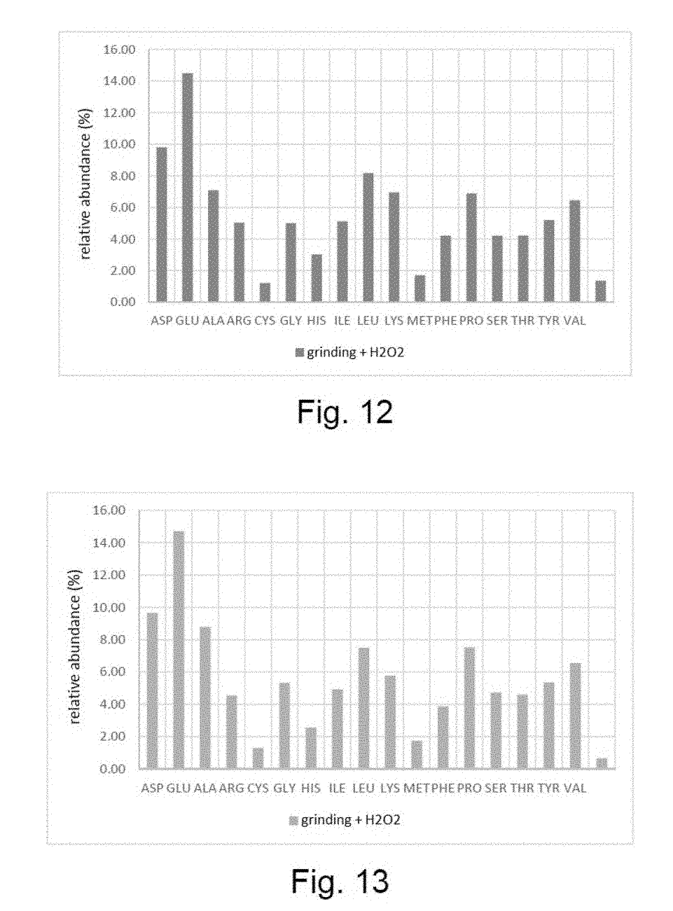

FIG. 12: Amino acid composition of the hydrolysate from a method with an oxidizing agent: TM+Neutrase,

FIG. 13: Amino acid composition of the hydrolysate from a method with an oxidizing agent: HI+Prolyve,

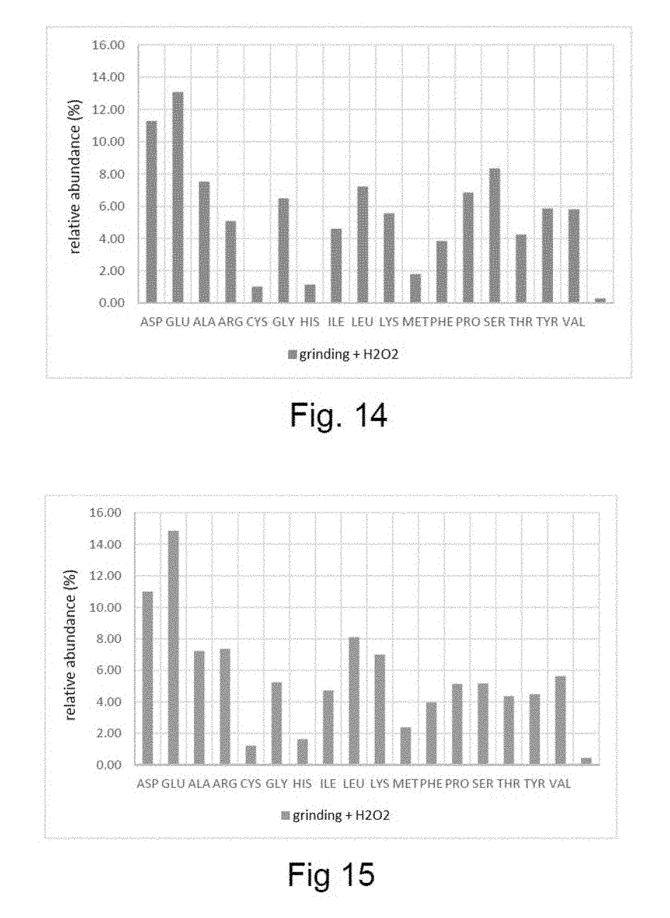

FIG. 14: Amino acid composition of the hydrolysate from a method with an oxidizing agent: GM+Prolyve,

FIG. 15: Amino acid composition of the hydrolysate from a method with an oxidizing agent: AD+Prolyve

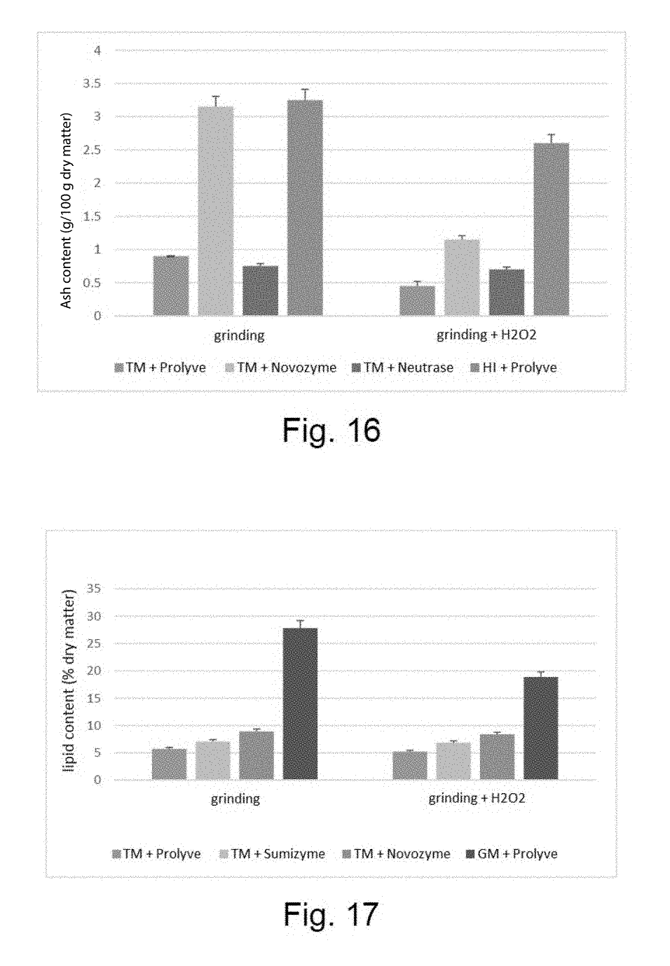

FIG. 16: Effect of the oxidizing agent on the ash content in chitin,

FIG. 17: Effect of the oxidizing agent on the lipid content in chitin,

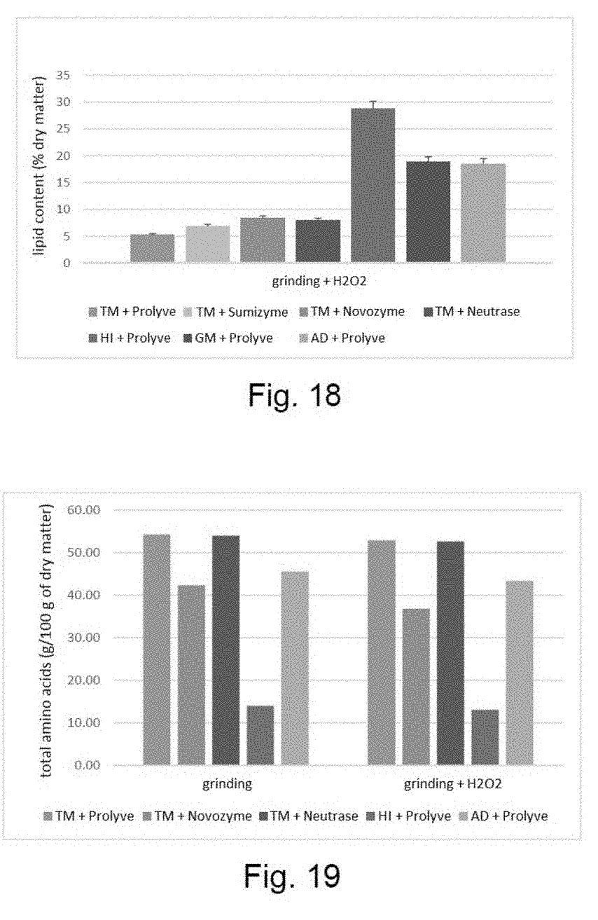

FIG. 18: Lipid content in chitin,

FIG. 19: Effect of the oxidizing agent on the amino acid content in chitin,

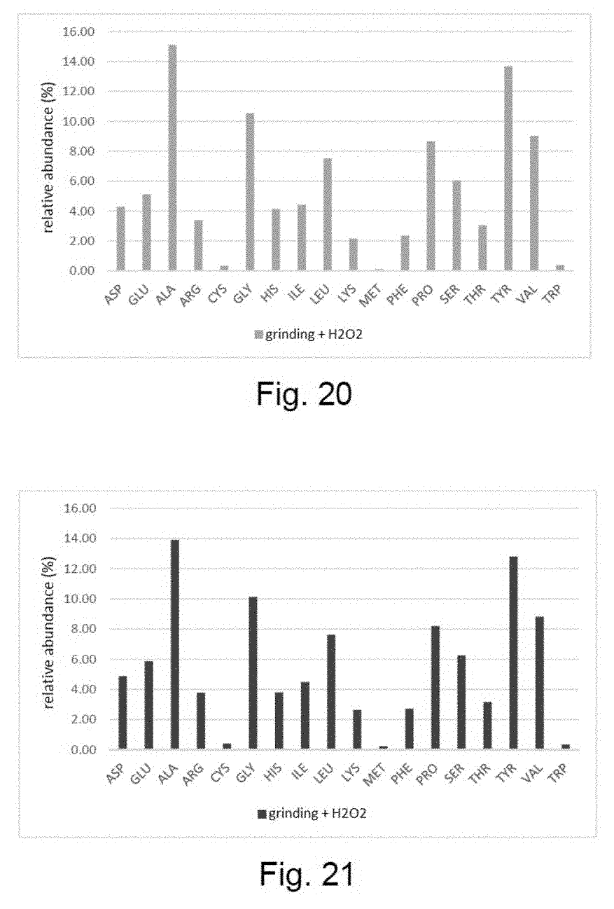

FIG. 20: TM+Prolyve: relative abundance of amino acids of chitin,

FIG. 21: TM+Sumizyme: relative abundance of amino acids of chitin,

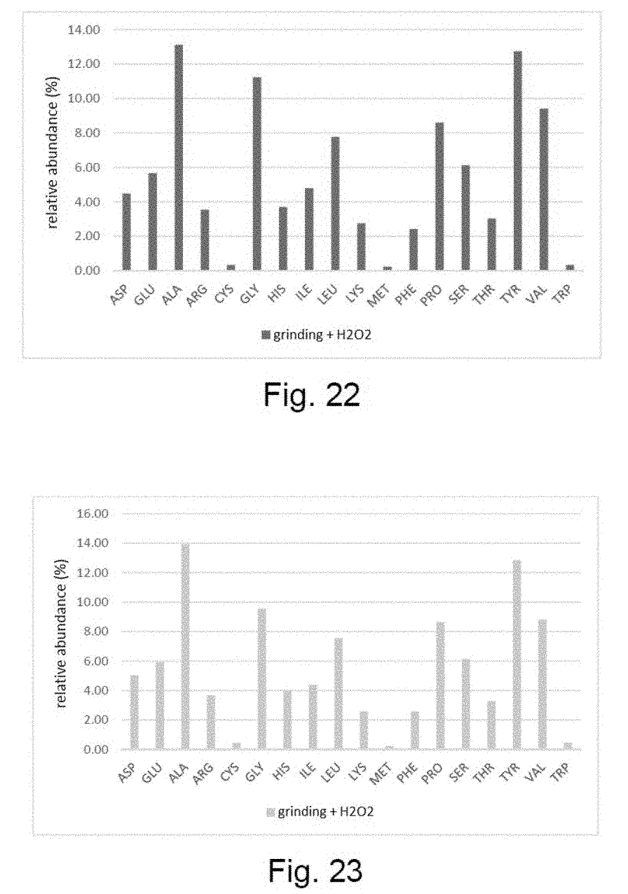

FIG. 22: TM+Novozyme: relative abundance of amino acids of chitin,

FIG. 23: TM+Neutrase: relative abundance of amino acids of chitin,

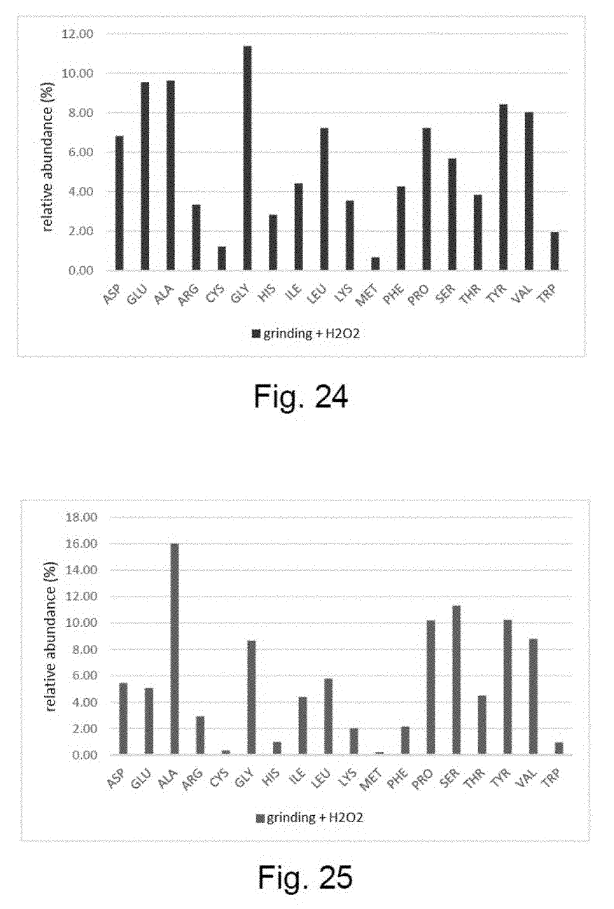

FIG. 24: HI+Prolyve: relative abundance of amino acids of chitin,

FIG. 25: GM+Prolyve: relative abundance of amino acids of chitin,

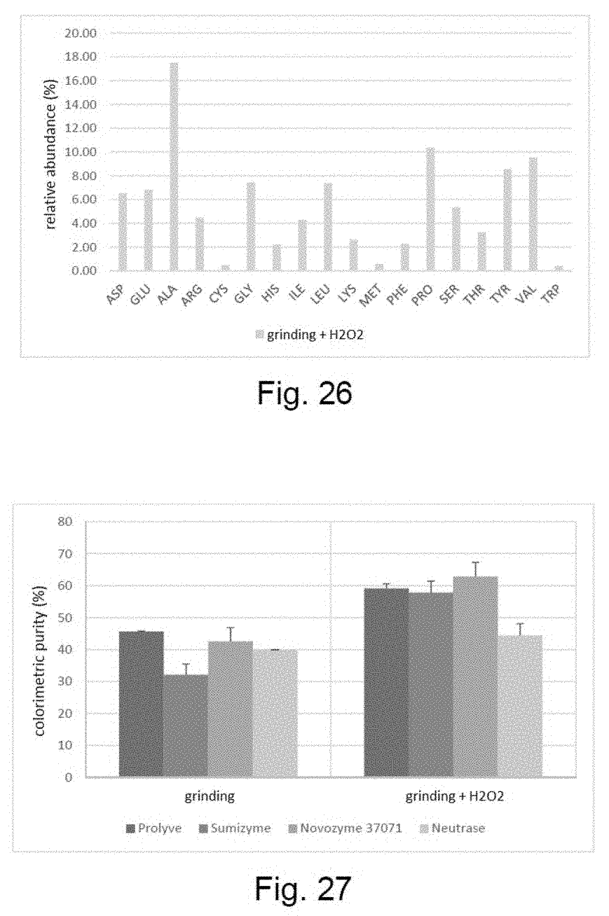

FIG. 26: AD+Prolyve: relative abundance of amino acids of chitin,

FIG. 27: Effect of the oxidizing agent on the colorimetric purity of chitin obtained from T. molitor-different enzymes,

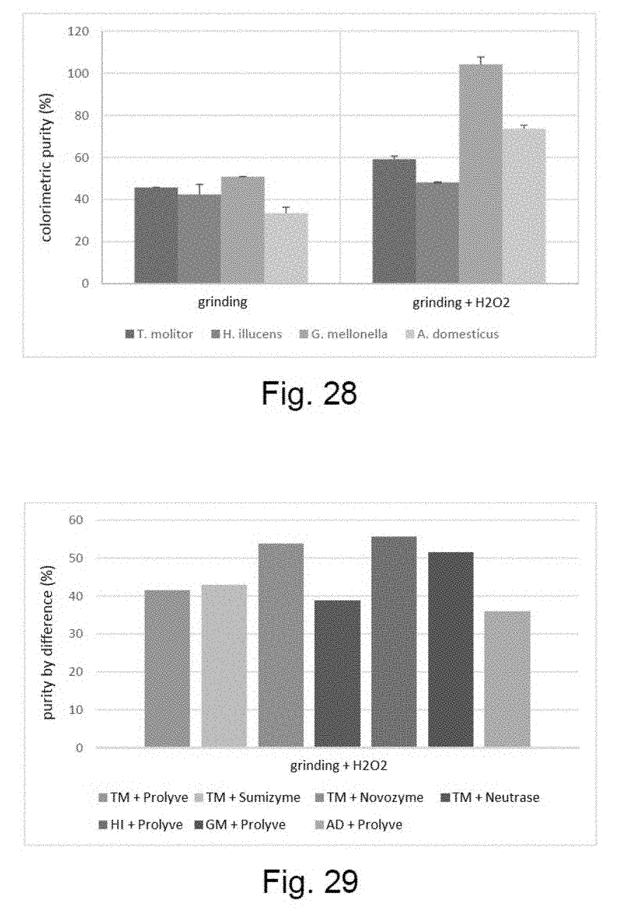

FIG. 28: Effect of the oxidizing agent on the colorimetric purity of chitin obtained with hydrolysis in the presence of Prolyve—different insects,

FIG. 29: Purity by difference of chitin obtained by different methods,

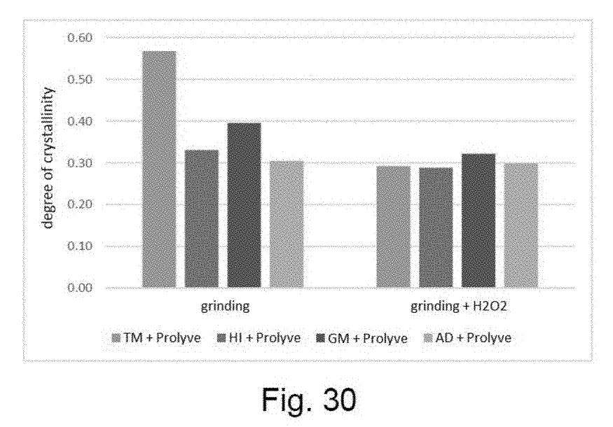

FIG. 30: Degree of crystallinity of chitins obtained.

### EXAMPLE 1: EXAMPLE OF A TREATMENT METHOD ACCORDING TO THE INVENTION

15 g of T. molitor larvae are introduced into a beaker, placed in a water bath at 100° C. containing 30 mL of a 6% solution of hydrogen peroxide brought to the boil beforehand. After 5 minutes, the beaker is removed from the water bath, the larvae are drained, and then ground with a volume of water of 15 mL. The liquid thus obtained is transferred to a 250-mL Erlenmeyer flask containing 50 mL of a 1% solution of protease (Prolyve), the whole is placed under magnetic stirring for 4 hours at 45° C. (pH approximately 6.5). The Erlenmeyer flask is then placed in a water bath at 90° C. for 15 minutes in order to deactivate the enzymes, and then the solution is filtered hot at 0.45-0.5 μm. The chitin thus obtained is dried for 24 hours at 70° C. This gives 0.6±0.05 g of chitin.

### EXAMPLE 2: INFLUENCE OF THE PRESENCE OF THE TREATMENT WITH OXIDANT IN THE METHOD ACCORDING TO THE INVENTION

25 g of T. molitor larvae are introduced into a beaker, placed in a water bath at 100° C. containing 50 mL of water brought to the boil beforehand. After 5 minutes, the beaker is removed from the water bath, the larvae are drained, and then ground with a volume of water of 25 mL. In the case of the reaction with hydrogen peroxide, the liquid thus obtained is placed in the presence of a solution of hydrogen peroxide for 1 hour, and then transferred to a 250-mL Erlenmeyer flask containing a 4% solution of protease (Sumizyme LP), or otherwise it is transferred directly to the Erlenmeyer flask containing the protease solution. The whole is placed under magnetic stirring for 4 hours at 45° C. (pH approximately 6.5). The Erlenmeyer flask is then placed for 15 minutes in a water bath at 90° C. in order to deactivate the enzymes, and the solution is then filtered hot at 0.45-0.5 μm. The chitin thus obtained is dried for 24 hours at 70° C.

The dry residue thus obtained after using hydrogen peroxide is 6.3±0.7% relative to the initial dry matter, whereas the dry residue resulting from a method without hydrogen peroxide is 9.75±0.9% relative to the initial dry matter.

The degree of purity of the chitin is determined compared to the weight of dry residue obtained relative to that resulting from chemical extraction, 5% of the initial dry matter. It is thus established at 79.9±9% for the product obtained after treatment with peroxide and at 51.5±4.9% in the absence of peroxide (see FIG. 2).

### EXAMPLE 3: INFLUENCE OF THE SEQUENCE OF THE DIFFERENT STEPS IN THE METHOD ACCORDING TO THE INVENTION

Obtaining Chitin Enzymatically (without Adding Oxidant)

50 g of T. molitor larvae are introduced into a beaker, placed in a water bath at 100° C. containing 50 mL of water brought to the boil beforehand. After 5 minutes, the beaker is removed from the water bath, the larvae are drained, and then ground with a volume of water of 100 mL. The liquid thus obtained is transferred to a 500-mL Erlenmeyer flask containing 150 mL of 1% protease solution (Prolyve), and the whole is placed under magnetic stirring for 4 hours at 45° C. (pH approximately 6.5). The Erlenmeyer flask is then placed for 15 minutes in a water bath at 90° C. in order to deactivate the enzymes, and the solution is then filtered hot at 0.45-0.5 μm. The chitin thus obtained is dried for 24 hours at 70° C. 1.656±0.021 g of chitin is obtained by this method.

Obtaining Chitin Enzymatically with Addition of H₂O₂ During Scalding

50 g of T. molitor larvae are introduced into a beaker, placed in a water bath at 100° C. containing 50 mL of a 6% solution of H₂O₂ in water brought to the boil beforehand. After 5 minutes, the beaker is removed from the water bath, the larvae are drained, and then mixed with a volume of water of 100 mL. The liquid thus obtained is transferred to a 500-mL Erlenmeyer flask containing 150 mL of 1% protease solution (Prolyve), and the whole is placed under magnetic stirring for 4 hours at 45° C. The Erlenmeyer flask is then placed in a water bath at 90° C. for 15 minutes in order to deactivate the enzymes, and then the solution is filtered hot at 0.45-0.5 μm. The chitin thus obtained is dried for 24 hours at 70° C. 1.98±0.22 g of chitin is obtained by this method.

Obtaining Chitin Enzymatically with Addition of H₂O₂ after Hydrolysis

50 g of T. molitor larvae are introduced into a beaker, placed in a water bath at 100° C. containing 50 mL of water brought to the boil beforehand. After 5 minutes, the beaker is removed from the water bath, the larvae are drained, and then ground with a volume of water of 100 mL. The liquid thus obtained is transferred to a 500-mL Erlenmeyer flask containing 150 mL of 1% protease solution (Prolyve), and the whole is placed under magnetic stirring for 4 hours at 45° C. (pH approximately 6.5). The Erlenmeyer flask is then placed in a water bath at 90° C. for 15 minutes in order to deactivate the enzymes, and then the solution is filtered hot at 0.45-0.5 μm. The residue is then placed in a 6% solution of H₂O₂ for 1 hour at 65° C. The chitin thus obtained is filtered (0.45-0.5 μm), and then dried for 24 hours at 70° C. 1.304±0.091 g of chitin is obtained by this method.

Obtaining Chitin Chemically

50 g of T. molitor larvae are introduced into a beaker, placed in a water bath at 100° C. containing 50 mL of water brought to the boil beforehand. After 5 minutes, the beaker is removed from the water bath, the larvae are drained, and then ground with a volume of water of 60 mL. The liquid thus obtained is transferred with 50 mL of water to a 1 L bottle. 500 mL of 1M HCl is added and the whole is stirred for 1 hour at 90° C. The reaction mixture is then filtered and washed with water until a clear residue is obtained. This residue is then transferred to a 1 L bottle, to which 500 mL of 1M NaOH is added, and the whole is stirred at 90° C. for 24 hours. The reaction mixture is then filtered and washed until a clear filtrate is obtained, and the residue is finally dried for 24 hours at 70° C. 0.944±0.005 g of chitin is obtained by this method.

Determination (by Viscometry) of the Molecular Weight of the Chitin Obtained

A bottle containing 1 g of chitin and 10 mL of 1M NaOH is held for 4 hours at 90° C. The mixture is then filtered (0.45-0.5 μm) and the residue thus washed is held for 24 hours at 70° C.

Preparation of the solvent: 5 g of LiCl is placed in 100 mL of N,N-dimethylacetamide, under stirring for 4-5 hours (until completely dissolved).

The stock solution is obtained by dissolving 0.2 mg of chitin in 1 mL of solvent. Dilutions with concentrations of 0.1 mg/mL, 0.08 mg/mL and 0.04 mg/mL are prepared from this stock solution. The viscosity of these various solutions is then measured in triplicate with a viscometer of the Ostwald type and the molecular weight is calculated from the formula:

[η]=KM_{w}^{α} (1)

with

[η]: intrinsic viscosity in cm³/g,

M_{w}: molar mass of the chitin in g/mol (or Da), and

the Mark-Houwink coefficients α=0.71 and K=0.000893,

the intrinsic viscosity being obtained from:

[η]=ηᵣ/C (2)

with

ηᵣ: reduced viscosity (without units),

C: concentration in mg/mL,

the reduced viscosity being obtained from:

ηᵣ=t/t₀ (3)

with

t: the falling time measured for the solution in s,

t₀: the falling time measured for the solvent in s.

It can be seen from FIG. 3 that the size of the chitin molecule obtained is a function of the method of extraction used. Thus, the chemical method damages the integrity of the molecule (M_{w} obtained is below 70000 g/mol), but the harshest treatment is that which consists of bleaching the chitin with hydrogen peroxide after hydrolysis, even enzymatic hydrolysis (M_{w} below 9000 g/mol).

The method according to the invention (enzymatic hydrolysis with addition of hydrogen peroxide during or just after scalding, i.e. at the beginning of the method) does decrease the size of the molecule relative to what can be found initially in the insect (M_{w} of chitin by simple enzymatic hydrolysis is 130000 g/mol), but to a much smaller extent (M_{w} close to 80000 g/mol) and the result obtained is greater than that associated with conventional chemical extraction.

### EXAMPLE 4: CHARACTERIZATION OF THE HYDROLYSATE AND THE CHITIN ACCORDING TO THE METHOD OF PRODUCTION USED

I. Material and Methods

a) Material

Insects

Various insects were studied:
a coleopteron: Tenebrio molitor (T. molitor or TM),
a lepidopteron: Galleria mellonella (G. melonella or GM),
a dipteron: Hermetia illucens (H. illucens or HI), and
an orthopteron: Acheta domesticus (A. domesticus or AD).

Enzymes

Various enzymes were used in the hydrolysis step.

This measurement of the enzymatic activity is based on the principle of measurement of tyrosine release at 275 nm during hydrolysis of casein by a proteolytic enzyme (Valley Research SAPU Assay method, by Karen PRATT).

$$\frac{SAPU}{g} = \frac{( Δ A - i ) \times 11}{m \times 30 \times C \times 1}$$

SAPU/g=one spectrophotometric unit of protease

ΔA=correlated absorbance

i=y-axis at origin

11=final reaction volume

M=slope of the calibration curve

30=reaction time (in minutes)

C=concentration of the enzyme (g/mL) in the enzyme solution added

1=1 mL volume of the enzyme solution added

The calibration curve is obtained by measuring the absorbance of tyrosine solutions of different concentrations in a phosphate buffer (0.2 M, pH 7).

5 mL of a solution of casein (0.7% w/v, phosphate buffer 0.2 M, pH 7, heated for 30 minutes at 90° C. and with 3.75 g/Lₛₒₗᵤₜᵢₒₙ added) is incubated with 1 mL of the enzyme solution (0.15 g in 100 mL of glycine buffer, 0.05M) to be tested at 37° C. for 30 minutes. Then 5 mL of TCA solution is added (18 g TCA, 11.35 g of anhydrous sodium acetate, 21 mL of glacial acetic acid, made up with demineralized water to 1 litre of solution), mix on a vortex, filter and measure the absorbance at 275 nm.

The control is prepared identically but without adding enzyme, 1 mL of demineralized water is added instead in order to have the same reaction volume.

The activities thus measured for the various enzymes used (Prolyve NP, Novozyme 37071, Neutrase and Sumizyme) are listed in Table 3.

TABLE 2
Correspondence of the activities and enzyme masses of
the enzymes used

| | enzyme | | | |
|---|---|---|---|---|
| | Prolyve | Novozyme | Neutrase | Sumizyme |
| Desired enzymatic | 3789.52 | 3789.52 | 3789.52 | 3789.52 |
| activity | | | | |
| Enzymatic | 3789.52 | 1662.35 | 2213.24 | 3237.19 |
| activity/g | | | | |
| m (g) | 1.00 | 2.28 | 1.71 | 1.17 |

b) Methods of Production

Method of Production with Grinding Only (Denoted “Grinding” in the Figures)

600 g of fresh insects (either larvae in the case of T. molitor, G. melonella or H. illucens; crickets in the case of A. domesticus) are introduced into a chamber, where they are killed with steam (115° C., 5 minutes). The insects are then introduced into a mixer and 75 mL of water is added per 100 g of insects, and the whole is then mixed. 100 g (wet weight) of product thus obtained is then introduced into a three-necked flask equipped with a condenser and a mechanical stirrer, and a proteolytic enzyme with an activity equivalent to 3789 SAPU is then added. The reaction is then heated to 45° C. for 4 hours. The temperature is then raised to 90° C. for 15 minutes, and the reaction mixture is finally filtered (0.40-0.45 μm). The residue is dried for 24 hours at 70° C.: this is therefore chitin obtained by the enzymatic route of purification; the filtrate is frozen and lyophilized: this is therefore the hydrolysate.

The method is identical whatever insect or enzyme is studied.

Method of Production with Grinding and Oxidizing Treatment of the Insect Cuticles (Designated “Grinding+H₂O₂” in the Figures)

600 g of fresh insects (whether larvae in the case of T. molitor, G. melonella or H. illucens; crickets in the case of A. domesticus) are introduced into a chamber, where they are killed with the vapour of a water/H₂O₂ mixture (6%) (115° C., 5 minutes). The insects are then introduced into a mixer and 75 mL of water is added per 100 g of insects, and the whole is then mixed. 100 g (wet weight) of the product thus obtained is then introduced into a three-necked flask equipped with a condenser and a mechanical stirrer, and a proteolytic enzyme with activity equivalent to 3789 SAPU is then added. The reaction is then heated at 45° C. for 4 hours. The temperature is then raised to 90° C. for 15 minutes, and the reaction mixture is finally filtered (0.40-0.45 μm). The residue is dried for 24 hours at 70° C.: this is therefore chitin obtained by purification by the enzymatic route; the filtrate is frozen and lyophilized: this is therefore the hydrolysate.

The method is identical whatever insect or enzyme is studied.

c) Analyses

Measurement of the Ash Content

The ash content was determined by the method based on EC Regulation 152/2009 dated 27 Jan. 2009.

Measurement of the Protein Content

The protein content is obtained by the Dumas method, with a conversion factor of 6.25, adapted from standard NF EN ISO 16634-1.

Measurement of the Lipid Content

The lipid content is obtained by a method adapted from EC Regulation 152/2009-method B-SN.

Pepsin Digestibility

Pepsin digestibility is measured by the method described in Directive 72/199/EC.

Relative Abundance of Amino Acids

The abundance of the amino acids was determined by a method derived from EC Regulation 152/2009 dated 27 Jan. 2009-SN. The tryptophan content was determined separately by a method adapted from EC Regulation 152/2009 dated 27 Jan. 2009-SN. The relative abundance was calculated by relating the content of each amino acid to the amino acid content.

Amino Acid Content

The amino acid content was determined by adding together the individual values obtained for each amino acid, including tryptophan.

Colorimetric Purity

The colour of the sample was estimated by analysing photographs of samples using the ImageJ software according to the three colours red, green and blue (RGB), their average representing an assessment of the true colour. A sample of prawn chitin marketed by Chitine France was taken as the standard (100% purity) and the colorimetric purity of the samples produced was calculated as a percentage of this colour (ratio of the colour of the sample to the colour of the standard).

Purity by Difference

For this measurement, the quantities of known impurities (amino acids, lipids and ash) were subtracted from the value of absolute purity (100%) to obtain the value of the purity estimated by difference; i.e. a sample that contains 30% proteins, 10% lipids and 1% ash is therefore assigned a purity of 100−30−10−1=59%.

Measurement of the Degree of Crystallinity

The measurements were carried out according to the WAXS (wide angle X-ray scattering) technique on Bruker D8 Advance apparatus (A25 DaVinci design) equipped with a Lynxeye XE detector. The results were interpreted following the method described in Loelovich, M. Res. Rev.: J. Chem. 2014, 3, 7-14.

II. Hydrolysate

a) Ash

Adding the oxidizing agent contributes to a decrease in the ash content in the hydrolysate, whatever the insects (FIG. 5) and the enzyme used (FIG. 4). This decrease can reach 20% when Novozyme is used and 23.4% when the experiment is carried out with Acheta domesticus (A. domesticus).

b) Protein Content

Adding the oxidizing agent makes it possible to increase the content of proteins in the hydrolysate (FIG. 6), in particular for insects rich in lipids, such as flying insects.

c) Lipid Content

Adding the oxidizing agent can have a significant effect on the lipid content in the hydrolysate (FIG. 7), and this decrease can reach close to 21% in certain cases (TM+Sumizyme).

d) Pepsin Digestibility

Digestibility is little affected by adding the oxidizing agent, for all the tests carried out, and the pepsin digestibility of the hydrolysates that have undergone treatment by adding the oxidizing agent in the method is above 93% whatever the insect or enzyme used (FIG. 8).

e) Abundance of Amino Acids

The hydrolysates resulting from the method with treatment with an oxidizing agent predominantly consist of aspartic acid, glutamate and proline, and to a smaller extent valine, lysine, leucine, serine and alanine, whatever the insect or enzyme studied (FIGS. 9-15).

III. Chitin

a) Ash

Adding the oxidizing agent also has a beneficial effect on the decrease in the ash content in the chitin obtained, whatever the insect or enzyme studied (FIG. 16). The decrease observed can thus reach 35.7% relative to the ash content present in the method without a pressing step.

b) Lipid Content

In contrast to the hydrolysate, adding the oxidizing agent has a significant effect on the lipid content of the chitin. In fact, as the oxidizing agent is added before grinding the insects, it essentially affects the tannins and the waxes present on the surface of the cuticle. The decrease can thus reach as much as 30% in certain cases (FIG. 17).

The lipid content of the chitin thus obtained is below 29% whatever the insect, and even below 8.5% in the case of T. molitor (FIG. 18).

c) Content and Relative Abundance of Amino Acids

Adding the oxidizing agent contributes slightly to a decrease in protein load of the chitin (FIG. 19).

Regarding the relative abundance of amino acids bound to chitin obtained from the method with an oxidative step, it can be stated that for all of the insects, alanine, tyrosine and proline, and to a smaller extent valine, glycine, leucine and serine are the main amino acids attached to the chitin, and their content is on average between 20 and 40% of the total amino acids (FIGS. 20 to 26).

d) Colorimetric Purity

Owing to its action on the tannins essentially present on the surface of the cuticles, the oxidizing agent plays an important role in improving the colorimetric purity of the chitin obtained, regardless of which insect or enzyme is studied (FIGS. 27 and 28). There is quite particularly marked improvement in the case of G. melonella, the purity finally obtained even exceeding 100%, i.e. greater than that of the commercial chitin used as the standard (FIG. 28).

e) Purity by Difference

Owing to its effect on the content of ash, lipids and amino acids in the chitin, the oxidizing agent plays an important role in improving the purity by difference of the chitin (FIG. 29).

f) Degree of Crystallinity

Treatment with an oxidizing agent tends to make the chitin more amorphous (FIG. 30), whatever insect is studied. The degree of crystallinity, i.e. the ratio of the crystalline and amorphous areas, of the chitin obtained is between 0.29 and 0.32.

1. Hydrolysate comprising at least 40% by weight proteins based on the total weight of dry matter, at a maximum 10% by weight ash based on the total weight of dry matter, and a water-soluble protein content larger than 12,400 g/mol less than 50%.

2. Chitin comprising an amino acid content less than or equal to 55% by weight based on the total weight of dry matter, an ash content less than or equal to 3.5% by weight based on the total weight of dry matter, and a purity by difference greater than or equal to 35% or a colorimetric purity greater than or equal to 44%.

3. Method for the production of chitin and/or chitosan from insect cuticles, comprising the following steps:
(i) treating the insect cuticles with an oxidizing agent, then
(ii) enzymatic hydrolysis of the insect cuticles with a proteolytic enzyme.

4. Method according to claim 3, in which the proteolytic enzyme is a protease.

5. Method according to claim 3 or 4, in which the oxidizing agent is selected from the group constituted by hydrogen peroxide, potassium permanganate, ozone and sodium hypochlorite.

6. Method according to any one of claims 3 to 5, comprising a step of killing the insects.

7. Method according to any one of claims 3 to 6, comprising a step of grinding the insects.

8. Method according to any one of claims 3 to 7, in which the oxidizing agent is hydrogen peroxide.

9. Method according to any one of claims 3 to 8, in which the insect or insects is/are selected from the group constituted by the Coleoptera, the Lepidoptera, the Orthoptera and the Diptera.

10. Method according to any one of claims 4 to 9, in which the protease is selected from the group constituted by aminopeptidases, metallocarboxypeptidases, serine endopeptidases, cysteine endopeptidases, aspartic endopeptidases, metalloendopeptidases.

11. Method for the production of chitin from insects, comprising the following steps:
a) killing the insects,
b) grinding the insects, grinding optionally being preceded or followed by a pressing step,
c) enzymatic hydrolysis of insect cuticles by a proteolytic enzyme,
d) recovery of the chitin,
the insect cuticles being treated with an oxidizing agent before step c).

12. Chitin obtainable by a method according to any one of claims 1 to 11.

13. Method for the production of chitosan from insects, comprising the following steps:
a) killing the insects,
b) grinding the insects, grinding optionally being preceded or followed by a pressing step,
c) enzymatic hydrolysis of insect cuticles by a proteolytic enzyme,
d) recovery of the chitin,
e) deacetylation of the recovered chitin,
f) recovery of the chitosan,
the insect cuticles being treated with an oxidizing agent before step c).
